(19) United States
(12) Patent Application Publication
(10) Pub. No.: US 2015/0118723 A1
(43) Pub. Date: Apr. 30, 2015

(54) Apparatus and method for generating eco-conscious products from waste

(71) Applicant: Derek Christopher Duzoglou, Miami, FL (US)

(72) Inventor: Derek Christopher Duzoglou, Miami, FL (US)

(21) Appl. No.: 13/998,407

(22) Filed: Oct. 30, 2013

(51) Int. Cl. C12P 7/64

(52) U.S. Cl. CPC C12P 7/64
USPC 435/134; 435/292.1

(57) ABSTRACT

An assembly for generating eco-conscious products from waste uses an anaerobic digester for anaerobically processing waste matter to produce anaerobic products selected from the group of products consisting of biogas, liquid digestate, solid digestate, and mixtures thereof One or more eco-assemblies receive the anaerobic products to generate at least one eco-conscious product. The eco-assemblies include one or more of an eco-matrix molder, an algae cultivator, a fast-pyrolysis chamber, a soil enhancer, a RDF pelletizer, and a hydroponic apparatus. A method is provided for generating eco-conscious products from waste by anaerobically processing waste matter to produce one or more of the anaerobic products, which are conveyed to one or more eco-assemblies to generate at least one eco-conscious product.

### FIELD OF THE INVENTION

Embodiments of the present invention are related to an apparatus and method for generating eco-conscious products. More specifically, embodiments of the present invention provide an apparatus and method for generating eco-conscious products from waste, such as municipal waste.

### BACKGROUND OF THE INVENTION

Catastrophic global trends indicate that the world continues to speed down a politically dangerous and economically unsustainable path—a path where natural resources are diminishing and the demands of an ever-expanding population are growing. As the world's population continues to grow exponentially, tremendous stress will be placed on society, the stabilization of governments, the environment, and the limited natural resources necessary for our survival.

The cost of energy has been dramatically increasing. Rising fuel costs, coupled with diminishing oil reserves have created a global economic strain. Worldwide energy consumption has gone up by 27% in the last 25 years and ironically, waste management operations in the U.S. alone require 300 million barrels of oil per year—seemingly a never-ending cycle.

The world's population has increased by an incredible 23% since 1990. Available land is becoming scarce and expensive and conventional landfill methods are at a tipping point. Essentially, food production will have to triple in order to sustain even current population growth rates.

The world is witnessing unprecedented health and environmental degradation. Toxic materials in landfills eventually leach into underground water sources threatening all human, animal and plant life throughout the world. As the largest human-created source of methane gas, landfills are also a significant contributor to global climate change.

The need for sustainable development is greater than ever, but any model of the past cannot be used as a pattern for the future. With the rapid increase of global population and the concomitant resource consumption, developing viable solutions is critical. Thus, what is needed and what has been invented is an apparatus and method for generating eco-conscious products from waste.

### SUMMARY OF EMBODIMENTS OF THE INVENTION

Embodiments of the present invention provide an assembly for generating eco-conscious products from waste matter, which may be any waste product or by-product, such as municipal waste, or the like. The assembly has an anaerobic digester for anaerobically processing the waste matter to produce anaerobic products, which are conveyed to one or more eco-assemblies to generate at least one eco-conscious product. The eco-assemblies comprise one or more of an eco-matrix molder assembly, an algae cultivator, a fast-pyrolysis assembly, a soil enhancer, a RDF pelletizer, and a hydroponic apparatus.

Embodiments of the present invention also provide a method for generating eco-conscious products from waste matter. The method includes anaerobically processing the waste matter to produce one or more anaerobic products, which are conveyed to one or more eco-assemblies to generate at least one eco-conscious product.

These provisions, together with the various ancillary provisions and features, which will become apparent to those skilled in the art as the following description proceeds, are attained by the embodiments and the methods and assemblies of the present invention.

### BRIEF DESCRIPTION OF THE DRAWINGS

FIG. 1 is a schematic flow diagram of an embodiment of the invention for generating eco-conscious products from waste.

FIG. 2 is a schematic flow diagram of an embodiment of the invention demonstrating the interconnectivity stemming from anaerobic digestion processing byproducts.

FIG. 3 is a schematic flow diagram of an embodiment of the invention demonstrating a biological mechanical assembly, which decomposes chemical structures of feedstock (e.g., waste feedstock), producing biogas, liquid digestate and solid digestate.

FIG. 4 is a schematic flow diagram of an embodiment of the invention illustrating biomass pyrolysis after receiving yard, wood waste and solid digestate; and plastics for pyrolysis after receiving plastic waste (e.g., MSW and SSC plastics).

FIG. 5 is a schematic flow diagram of an embodiment of the invention demonstrating a biological mechanical or structural assembly having a fast-pyrolysis chamber or reactor for producing bio char, bio oil, and syngas (or synthesis gas) which are recycled for heating the fast pyrolysis process.

FIG. 6 is a schematic flow diagram of an embodiment of the invention illustrating eco-matrix molding after receiving liquid, solid digestates, and bio char; and after receiving paper and cardboard (e.g. MSW and SSC paper and cardboard).

FIG. 7 is a schematic flow diagram of an embodiment of the invention demonstrating an eco-matrix mechanical or structural assembly which produces organic composites from liquid and solid digestate, bio char and paper and cardboard (e.g., MSW and SSC paper and cardboard).

FIG. 8 is a schematic flow diagram of an embodiment of the invention illustrating algae cultivation receiving liquid digestate, bio char, biogas, micronutrients, and algae strain.

FIG. 9 is a schematic flow diagram of an embodiment of the invention demonstrating an algae-cultivation mechanical or structural assembly which produces bio oil, nutra/pharmaceuticals and and/or chemical feed stock.

FIG. 10 is a schematic flow diagram of an embodiment of the invention illustrating a RDF pelletizer receiving wood, yard wastes and paper & cardboard waste; and receiving and solid digestate.

FIG. 11 is a schematic flow diagram of an embodiment of the invention illustrating anaerobic digestion receiving food waste, yard waste, and agricultural waste to produce biogas, liquid and solid digestates.

FIG. 12 is a schematic flow diagram of an embodiment of the invention illustrating fast-pyrolysis (e.g., in a fast-pyrolysis chamber or reactor) producing bio oil, bio char, and syngas after receiving for pyrolysis purposes wood waste, food waste, yard waste, agricultural waste, plastic waste, and solid digestate.

FIG. 13 is a schematic flow diagram of an embodiment of the invention illustrating eco-matrix molding (e.g., in an eco-matrix molder) producing organic composites, after receiving paper & cardboard waste and wood waste. FIG. 1 illustrates that in addition to paper & cardboard waste and wood waste, liquid and solid digestates, and biochar are received for producing organic composites.

FIG. 14 is a schematic flow diagram of an embodiment of the invention illustrating algae cultivation (e.g., in an algae cultivator), producing bio oil, chemical feedstock, and nutra/pharmaceuticals after receiving algae strain, micro nutrient, water source, and carbon dioxide source.

FIG. 15 is a schematic flow diagram of an embodiment of the invention illustrating refuse derived fuel [i.e. for pelletizing] for producing RDF pellets and carbon credits after receiving other waste, wood waste, yard waste, and paper & cardboard.

FIG. 16 is a schematic flow diagram of an embodiment of the invention illustrating hydroponics (e.g., in a hydroponics chamber or reactor), producing organic produce after receiving seed supply, micro nutrients, water source and light source.

FIG. 17 is a schematic flow diagram of an embodiment of the invention illustrating organic soil [amendments] enhancers producing solid soil amendments after receiving compost, peat, manure, slurry, seaweed, algae, and animal wastes.

### DETAILED DESCRIPTION OF PREFERRED EMBODIMENTS OF THE INVENTION

In the description herein, numerous specific details are provided, such as examples of components and/or methods, to provide a thorough understanding of the embodiments of the present invention. One skilled in the relevant art will recognize, however, that an embodiment of the invention may be practiced without one or more of the specific details, or with other apparatus, systems, assemblies, methods, components, materials, parts, and/or the like. In other instances, well-known structures, materials, or operations are not specifically shown or described in detail to avoid obscuring aspects of the embodiments of the present invention.

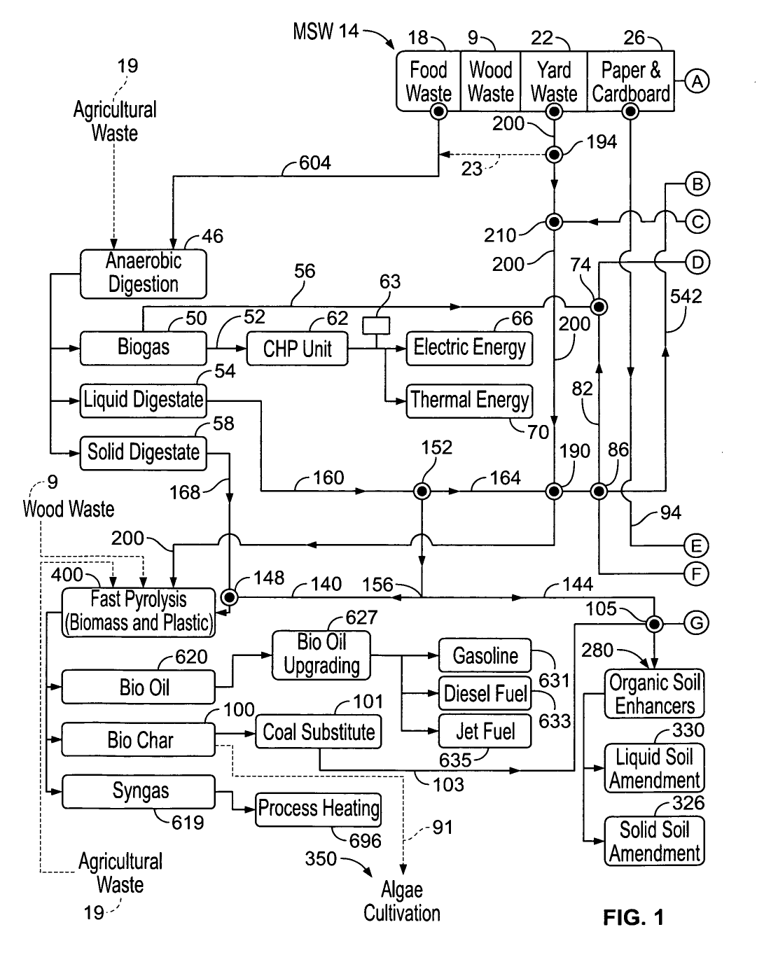

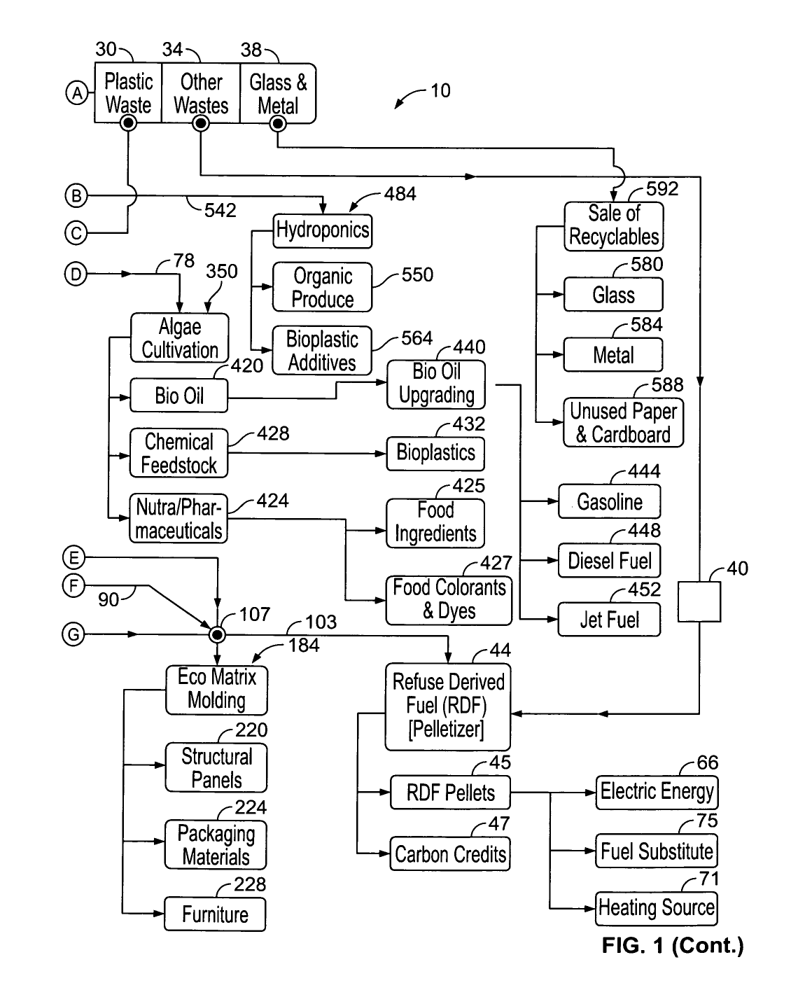

Referring in detail now to the drawings, wherein similar parts of the invention are identified by like reference numerals, there is broadly illustrated in FIG. 1 a schematic diagram, generally illustrated as 10, which is a schematic flow diagram for sustainably re-engineering existing solid waste to generate eco-conscious products. The schematic flow diagram 10 is based on conversion methods which operate without the introduction of outside/ambient air, relying only on the oxygen in the waste itself—as in pyrolysis which is a thermo-chemical decomposition of organic material at elevated temperatures. It involves the simultaneous change of chemical composition and physical phase, and is irreversible. Gas-containing oxygen is not injected or included in any of the conversion steps. The oxygen needed for the conversion methods originates from the waste that is being converted.

Solid waste includes wastes such as garbage, industrial waste, sewage sludge, ashes, discarded metal, trash, and any solid or semi-solid material, or any of the like. In a preferred embodiment of the invention, after waste is delivered to the re-engineering solid-waste location, the waste will be taken to one of two receiving areas—source separated components (SSC) or municipal solid waste components (MSWC). The source separated components (SSC) include comingled recyclable materials (i.e., paper, cardboard, plastics, food waste, wood waste, liquid substrates, and yard/agricultural waste), while municipal solid waste components (MSWC) typically include the solid waste, or garbage, collected from the residents of a city and is composed of mostly paper, plastic, glass, metal, food scraps and other household wastes, all which are typically disposed of in a landfill.

Both source separated components (SSC) and municipal solid waste components (MSWC) require further processing. Source separated components (SSC) are placed through a pre-screening process to remove any contaminants or additional comingled materials. Municipal solid waste components (MSWC) undergo a much more detailed mechanical and manual sorting process to separate the municipal solid waste into various waste components, e.g., food waste, paper, cardboard, plastic, glass, metal and miscellaneous materials. Municipal solid waste is generally identified in the schematic flow diagram of FIG. 1 by “14.”

Referring to FIG. 1 for discussing various preferred embodiments of the invention, municipal solid waste 14 may be obtained and transported by any suitable means, such as by garbage trucks or the like. The municipal solid waste 14 may have any various sources, such as residential, commercial, or agriculture. As best shown in FIG. 1, in a preferred embodiment of the invention, municipal solid waste 14 comprises food waste 18, yard waste 22, wood waste 9, paper and cardboard 26, plastic waste 30, other wastes 34, and glass and metal 38. The various waste components of the municipal solid waste 14 (i.e., food waste 18, yard waste 22, wood waste 9, paper and cardboard 26, plastic waste 30, other wastes 34, and glass and metal 38) may have been previously separated from the municipal solid waste 14 before being transported to the re-engineering solid waste location (broadly illustrated in FIG. 1 as 10) by any suitable-waste transporting means (e.g., garbage truck or the like), or the respective waste components may be separated after discharge from the suitable-waste transporting means. Any suitable means may be employed for preparing the respective waste components for being processed by the various assemblies or plants at the re-engineering solid waste location, such as shredders or the like.

Food waste 18 includes food which is broadly any nutritious substance that people or animals eat or drink, in order to maintain life and growth. More specifically, and is well known, “food” is any nutritious substance consumed to provide nutritional support and includes essential life-preserving nutrients, such as proteins, vitamins, minerals, carbohydrates, fats, and other matter. Yard waste 22 and wood waste 9 may be any waste from a yard, field, or the like, such as grass clippings, leaves, weeds, plants, tree limbs, or any of the like.

Paper/cardboard 26 has its genesis from complex carbohydrates, (C₆H₁₀O₅) ₙ, that is composed of glucose units or pulp derived mainly from wood, rags, and certain grasses. Paper is processed into flexible sheets or rolls by deposit from an aqueous suspension. Cardboard, as well known, is thick paper that is made of pressed paper pulp or pasted sheets of paper.

Plastic waste 30 comprises any of various organic compounds produced by polymerization, capable of being molded, extruded, cast into various shapes and films. Well-known plastics are polyethylene, polypropylene, and polyvinylchloride.

Glass and metal 38 include glass and metal and any associated matter. Metal may be any metal, such as those classified as ferrous (i.e. iron), non-ferrous (e.g. aluminum, copper, lead, zinc, tin) or alloys (a mixture of two or more metals). Glass may be any type of glass, such as those from a class of materials that solidify from the molten state, and are typically made by silicates fusing with an oxide, such as aluminum oxide or boric oxide. Municipal glass is typically transparent or translucent and brittle. As best shown in FIG. 1, glass and metal 38 may be separated into components: glass 580, metal 584, and unused paper/cardboard 588 for recycle sale 592.

In a preferred embodiment of the invention, as previously indicated, other waste 34 may be any other type of waste, preferably waste which is capable of producing refuse derived fuel (RDF), or solid recovered fuel/specified recovered fuel (SRF). SRF can be distinguished from RDF in the fact that it is produced to reach a standard such as CEN/323 ANAS.

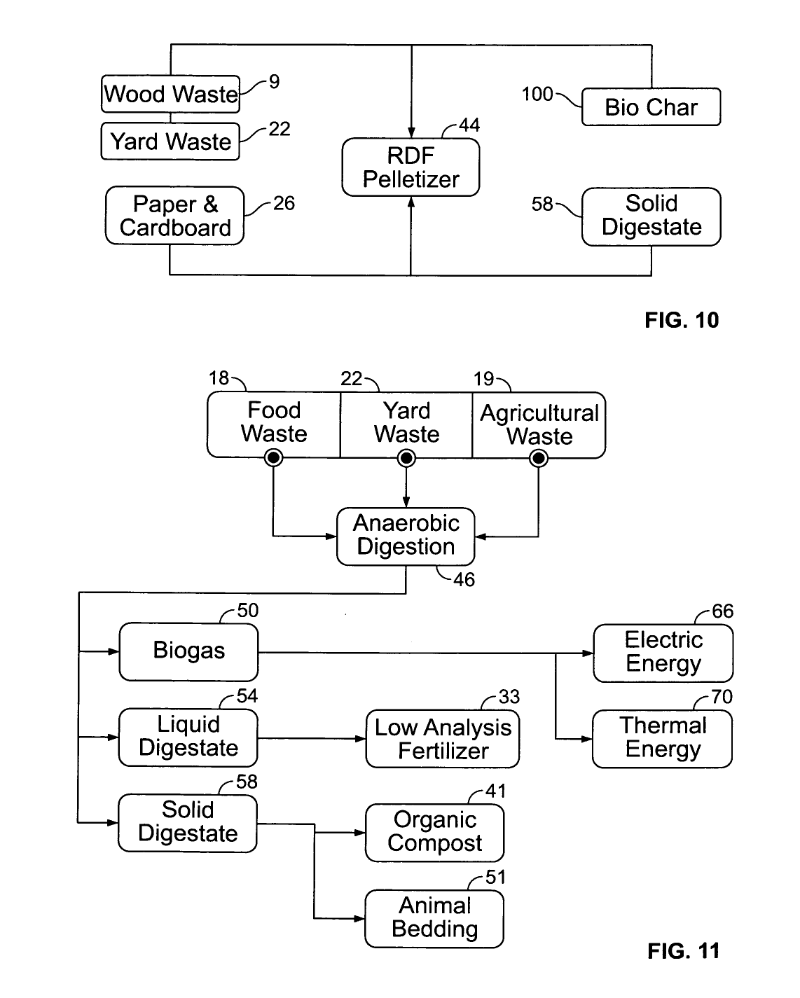

As best shown in FIGS. 1 and 15, RDF is produced from largely combustible one or more of (or components of) other waste 34, wood waste 9, yard waste 22 and paper & cardboard 26, as well as bio char 100 and solid digestate 58 (see FIG. 10). These waste streams are typically capable of producing RDF pellets 45 (from which can be generated electric energy 66, fuel substitute 75 and heating source 71), and carbon credits 47. In one embodiment of the invention, RDF is produced by shredding and dehydrating other wastes 34, such as in an RDF-preparation plant 40 (best shown in FIG. 1) employing conventional waste converting technology.

In preferred embodiments of the present invention, production of RDF is broken into three stages: (1) separation; (2) drying & shredding; and (3) pelletizing. During the separation phase there are three steps which include visual inspection/manual separation, screening and magnetic separation. Visual inspection deals with the manual removal of unwanted components, screening is responsible for sorting the RDF-viable waste by size, and the magnetic separation removes any metallic waste present in the feedstock. The resulting separation of incombustible & recyclable materials (not usable as RDF) is broken down into ferrous metals, non-ferrous metals, glass, textiles and bulky waste.

The drying and shredding stage is preferably repeated at least twice in order to ensure the necessary requirements of the final RDF are met. Through drying, the moisture content of the waste is reduced between about 60 to about 80%. Subsequent pre-shredding and secondary shredding will reduce the bulk waste feedstock from about 0.50″ to about 1.00″ particle size.

Pelletization, the final stage, begins with a process known as air classifying where less dense materials are collected. Basic setups run compressed air at a pressure of about 100 psig and with a flow rate of about 5 scfm (standard cubic feet per minute)—while more complex systems will operate at higher flow rates and pressures to accommodate for the variations in waste stream composition. Upon collection of the appropriate material, the remaining waste is compacted into its specified shape—referred to as pelletizing.

In FIG. 1, component other waste 34 is shown flowing to plant 40. RDF-preparation plant 40 uses mechanical heat treatment, mechanical biological treatment, or waste autoclaves which may employ pressurized steam. Plant 40 may also remove and/or reduce pollutants and/or heavy metals. From plant 40 RDF other waste 34 is sent to RDF pelletizer 44 for producing RDF pellets 45 and carbon credits 47. Electric energy 66, fuel substitute 75 and heating source 71 may be produced from RDF pellets 45.

Separate from producing electric energy 66, fuel substitute 75 and heating source 71, RDF may be used in a variety of ways, such as alongside traditional sources of fuel in coal power plants, as well as in the cement kiln industry. RDF can also be fed into plasma arc gasification modules, pyrolysis plants and where the RDF is capable of being combusted cleanly or in compliance with the Kyoto Protocol, RDF can provide a funding source, such as when carbon credits 47 are sold on the open market via a carbon exchange.

The term “conduit” in certain instances hereafter will be used for purposes of describing how products or matter (i.e., any liquid, solid or gas) of the present invention are sent, transmitted, or conveyed from one location to another. When the term “conduit” is stated in the specification and/or the claims, the meaning of “conduit” is not to be limiting to any particular means for sending, transmitting or conveying products or matter, but is meant to mean any means, such as by way of example only, pipe, tube, channel, conveyor, duct, chute, or any of the like.

In another preferred embodiment of the invention, in the case of if/when there is left over or underutilized waste streams aside from other wastes 34, as best shown in FIG. 10, yard waste 22, wood waste 9, and paper and cardboard 26 may be used to generate RDF. The paper/cardboard 26 is sent to RDF pelletizer 44 through conduit 94 (valve 86 is closed), through valve 107 (valve 105 is closed) and then through conduit 103. The yard waste 22 and wood waste 9 are sent to RDF pelletizer 44 as a mixture with paper/cardboard 26. More specifically, yard waste 22 and wood waste 9 are mixed and transmitted through conduit 200, through valve 194, through valve 210 (set to only allow the passage of yard waste 22), through valve 190 and toward valve 86 (valve 152 is closed), through valve 86 and then valve 107 (valve 105 is closed) and then through conduit 103.

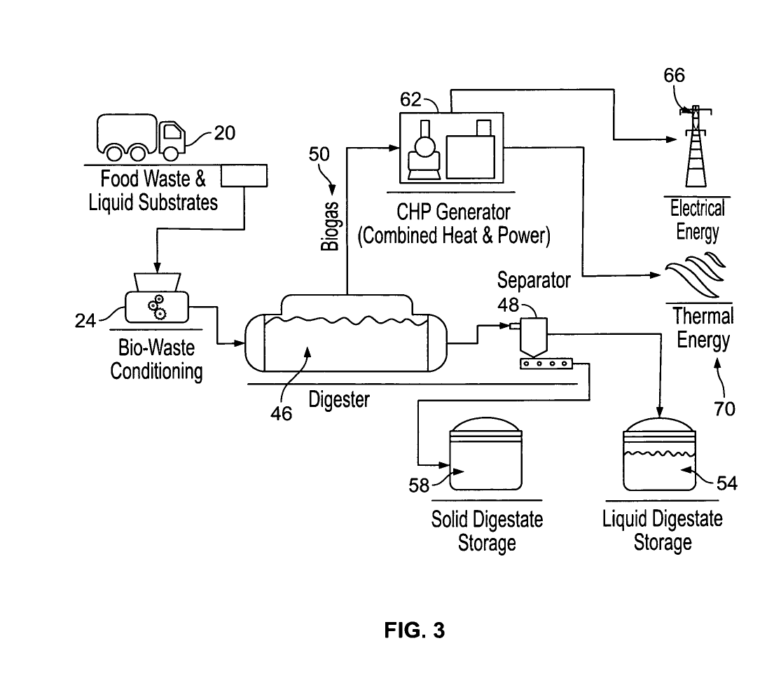

As further best shown in FIG. 10, in another embodiment, RDF may be produced from the solid digestate 58, and from bio char 100—charcoal created by fast pyrolysis 400 of biomass (see FIG. 1). Biomass is known to be biological material derived from living, or recently living organisms (e.g., plants or plant-derived materials that are specifically called lignocellulosic biomass). The bio char 100 may be used directly as a coal substitute 101, or the subsequently produced coal substitute 101 may be sent through conduit 103 to the RDF pelletizer 44, after passing through valves 105 and 107. Solid digestate 58 (produced in anaerobic-digestion tank 46 of FIG. 3) is sent to RDF pelletizer 44 through conduit 168, through valve 148, through conduit 140, through conduit 144 (valve 152 is closed), through valve 105 which is set to allow the passage of both solid digestate 58 and bio char 100, and then subsequently through valve 107 and into RDF pelletizer 44. Bio char 100 lends itself not only to applications as a component in the refuse derived fuel (RDF) [pelletizer], but also in applications as an organic soil enhancer/amendment 280, and/or as an additive in the eco-matrix molding 184 process.

An RDF preparation assembly, such as RDF-preparation plant 40, systematically prepares the yard waste 22, wood waste 9, paper and cardboard 26, solid digestate 58 and bio char 100 prior to introducing these components or mixtures into RDF pelletizer 44. For this embodiment of the invention, after the pre-processing/preparation stage in plant 40, the resulting yard waste 22, wood waste 9, paper/cardboard 26, bio char 11, solid digestate 58, and other, miscellaneous waste 34, are introduced into the RDF pelletizer 44 where it is processed into RDF pellets 45 and carbon credits 47, as best shown in FIGS. 1 and FIG. 15. It is known that carbon credits 47 is a permit that allows the holder to emit one ton of carbon dioxide or greenhouse gas with a equivalent to one ton of carbon dioxide. The RDF pelletizer 44 physically compresses the various feed stocks (i.e., wood waste 9, yard waste 22), paper/cardboard 26, bio char 100 and solid digestate 58) to produce RDF in three forms: pellets 45, bricks and logs (not shown), all of which may be used for other purposes, either stand-alone or in a recursive recycling process. Only pellets 45 are shown in FIGS. 1 and 15.

In a preferred embodiment of the invention, one or more of all feed stocks (e.g., by way of example, one or more components: MSW 14, solid digestate 58, bio char 100, and organic waste 15) prior to being conveyed to RDF [pelletizer] 44 for producing products from waste streams are dehydrated to essentially remove all water and moisture. This is typically accomplished via a waste autoclave (not shown in the drawings). A waste autoclave is a form of solid waste treatment that utilizes heat, steam and pressure to properly treat the waste for RDF production, which can be done in batches or a continuous-flow process. In a batch process, saturated steam is pumped into the autoclave at temperatures around 320 degrees F. The steam pressure in the vessel is maintained up to about 6 bar gauge for a period of up to about 45 minutes to allow the process to fully ‘cook’ the waste—subsequently reducing the bulk volume of the waste by about 60%. By way of example only, if essentially all of the moisture content in MSW 14 (and solid digestate 58, bio char 100, and organic waste 15) is removed, from about 600 lbs to about 2,600 lbs of RDF pellets 45 are produced, more preferably from about 900 lbs to about 2,300 lbs, most preferably from about 1,200 lbs to about 2,000 lbs (e.g. about 1,600 lbs of RDF pellets 45 per ton of waste).

It is to be understood that the embodiment of the invention illustrated in FIGS. 1, 10 and 15, and/or as discussed above, for regulating the flow, the direction, and/or the sending or transmission of yard waste 22, wood waste 9, paper/cardboard 26, bio char 100 and solid digestate 58 to RDF pelletizer 44 for pelletizing RDF is exemplary, not limiting. In the spirit and scope of the present invention, the yard (and wood 9) waste 22, paper and cardboard 26, solid digestate 58 and bio char 100 may come from any source (e.g., any municipal solid waste and/or organic waste 15), other than sources indicated in FIG. 1. Thus, it is to be understood that the spirit and scope of the present invention for producing the RDF from yard waste 22, wood waste 9, paper/cardboard 26, bio char 100 and solid disgestate 58 includes other variations and modifications of that illustrated in FIGS. 1, 10 and 15, and/or as discussed above.

In addition to the embodiments of the invention illustrated in FIGS. 1, 10 and 15, it is also to be understood that for any and all embodiments of the invention described herein, and/or illustrated in all other Figures (other than FIGS. 1, 10 and 15), for regulating the flow, the direction, and/or the sending or transmission of all products, by-products, or components, are exemplary, not limiting. Thus, any arrows in the drawings/figures should be considered only as exemplary, and not limiting, and are to include any and all other variations and modifications of those embodiments illustrated in all other Figures (other than FIGS. 1, 10 and 15), and/or as discussed hereafter.

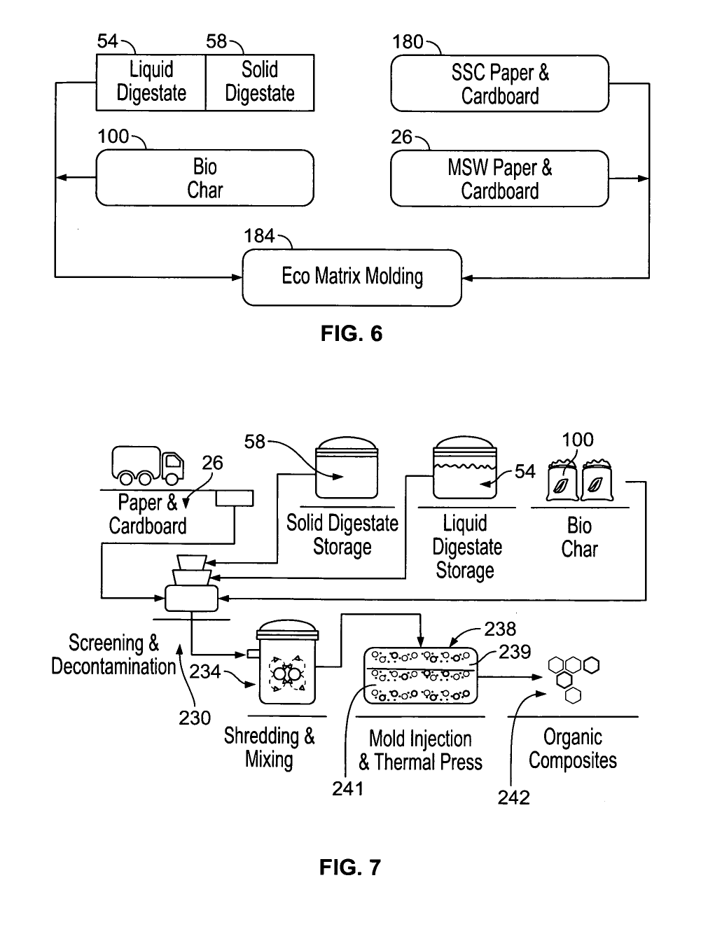

Valve 107, as indicated, regulates the metering or flow of various components and mixtures. In the embodiment of the invention illustrated in FIGS. 1, 6 and 7, for eco-matrix molding by eco-matrix molding 184, valve 107 controls the flow of bio char 100, MSW and SSC paper/cardboards, respectively 26 and 180, and liquid and solid digestates, respectively 54 and 58. Bio char 100 (coal substitute 101) flows through conduit 103 and through valves 105 and 107 and then to eco-matrix molding 184. MSW paper/cardboard 26 flows to eco-matrix molding 184 through conduit 94 and subsequently through valve 107. Liquid digestate 54 flows through conduit 160, valve 152, conduit 164, through valves 190 and 86, through conduit 90 and then through value 107. Component SSC paper/cardboard 180 may have any suitable source, such as recyclable materials (i.e., paper, cardboard, plastics, food waste packaging).

Referring now to FIGS. 7 and 13 for a preferred embodiment for eco-matrix molding 184, once the liquid and solid digestates 54 and 58, bio char 100, paper/cardboard waste streams 26 (i.e., the SSC and MSW paper/cardboard 180 and 26, respectively), and wood waste 9 (see FIG. 13, not shown in FIG. 7) (wood waste 9 in FIG. 1) have been delivered to the eco-matrix molding location, they are placed into a secondary screening and contaminant removal process, generally illustrated as 230 and best shown in FIG. 7. As soon the waste-stream mixture has been cleaned, the admixed waste streams are mixed and placed in a shredder/mixer 234 where particle size is dramatically reduced, producing an organic fiber mix. This mixture is then combined with water to create a pumpable matrix. The matrix is then pumped into a mold injection/thermal press assembly, generally illustrated as 238, having a mold injection 239 and a thermal press 241.

After the pumpable matrix enters mold injection/thermal press assembly 238, organic fiber particles sink to the bottom and “coat” the mold 239. After the desired quantity of matrix has been applied, the water is drained, leaving behind the mold 239 now covered with a thick layer of organic fiber. The water is drained using a specialized filtration system (filters with a minimum rating of about 50 microns) to ensure that all fiber remains within the mold. Upon removal of the mold 239, a component called a compression plate (not shown) is placed over it in order to lock all fibers in place and prevent any shifting during the curing process. The mold 239 is then placed into a thermal press 241, where process heat (thermal energy 70) produced during the anaerobic digestion phase 46 is applied to cure, or bake, the mold 239. Heat and pressure create a special natural bond. The thermal press 241 operates ideally at a temperature of about 500 degrees F. and a pressure of about 200 psi. Application time of the thermal press 241 depends heavily on the specifications of the component being created, and may vary accordingly.

Compared to a dry process, eco-matrix molding 184 of the present invention has the added benefit of achieving cost effectively better fiber adhesion, panel strength and product uniformity. The mold injection/thermal press assembly 238 produces organic composites 242 from which by-products may be produced. The by-products produced may be any desired by-products, such as structural panels 220, packaging materials 224, and furniture 228, as best shown in FIGS. 1, 7 and 13. The eco-matrix molding process of the present invention produces lighter weight products that use no toxic additives, from a much broader range of raw material sources. By way of example only, a matrix of paper and cardboard (which is the resulting fiber mix after screening, decontamination and shredding derived from MSW and/or SSC paper/cardboard—26 and 180 respectively) can yield from about 25,000 sq. feet per ton of processed paper/cardboard to about 70,000 square feet per ton of processed paper/cardboard of structural panels 220 which may be of any shape, more preferably from about 30,000 square feet per ton to about 65,000 square feet per ton, most preferably from about 40,000 square feet per ton to about 60,000 square feet per ton (e.g., 50,000 square feet per ton of processed paper/cardboard per structural panels 220). Thus, a matrix of paper and cardboard can yield about 50,000 square feet of structural panels 220 with dimensions of 4′×8′×⅜″ (a cubic foot).

In the embodiment of the invention illustrated in FIGS. 1 and 17, there is shown an organic soil enhancer, generally illustrated as 280, which enhances or modifies and processes naturally occurring organic fertilizer into processed high-grade organic fertilizers which may be used commercially for agriculture purposes. Organic fertilizers are naturally occurring fertilizers (i.e. compost, manure). Naturally occurring organic fertilizers more specifically include manure, slurry, worm castings, peat, seaweed, humic acid and guano. Processed organic fertilizers include compost, blood meal, bone meal, humic acid, amino acids, and seaweed extracts. Other examples are natural enzyme-digested proteins, fishmeal, and feather meal. Decomposing crop reside (green manure) from prior years is another source of fertility. Although the density of nutrients in organic material is comparatively modest, they have many advantages. The majority of nitrogen-supplying organic fertilizers contain insoluble nitrogen and act as a slow-release fertilizer. By their nature, organic fertilizers increase physical and biological nutrient storage mechanisms in soils, mitigating risks of over-fertilization.

In one of the preferred embodiments of the present invention, the materials to be enhanced or amended include one or more of bio char 100, liquid digestate 54 and solid digestate 58. The digestates (i.e., liquid and solid digestates) originate as by-products from anaerobic (non-aerobic) digestion 46. Bio char 100 is a by-product from fast pyrolysis 400. The solid digestate flows through conduit 168, through valve 148, through conduit 140, juncture 156 where they may be admixed with liquid digestate 54 which originated after flowing through conduit 160, and through valve 152 (valve 190 is closed) to juncture 156. After the solid and liquid digestates, 58 and 54 respectively, have been admixed with bio char 100 (coal substitute 101) in slurry (water basis) form, the mixture flows through valve 105 for being enhanced at or in organic soil enhancers 280.

In a preferred embodiment of the invention for converting or processing naturally occurring fertilizer (or solid/liquid digestates, bio char) into processed organic fertilizers, as best shown in FIG. 17, the enhancing-feed stock for organic soil enhancer 280 includes compost 290 (decomposed organic matter), peat 294 (partially carbonized vegetable matter), manure 298 (animal excrement and droppings), and slurry 302 (a semi-liquid mixture, typically of fine particles of manure, or coal suspended in water).

As previously indicated, the coal or coal substitute for a water-based slurry may also originate from the bio char 100 which has been converted into coal substitution 101 and which is subsequently passed through conduit 103 to valve 105 which meters or controls the flow of the coal substitute 101 into the organic soil enhancer 280. The feed stock for organic soil enhancer 280 also includes seaweed 310, algae 314 and animal waste 318 (feces or manure, such as that which produces hydrogen sulfide, methane, ammonia, and carbon dioxide).

After these feedstocks are introduced into the organic soil enhancer 280 and processed, amendments produced include solid soil amendments 326 and liquid soil amendments 330 (liquid material that produces the same results of solid soil amendments 326). In the preferred embodiment of the invention illustrated in FIG. 17, solid soil amendments 326 include, by way of example only, compost 336, mineral fertilizers 340, plant based fertilizers 344, and animal based fertilizers 348, and mixtures thereof.

After any feedstock (e.g. solid/liquid digestates, coal char) has flowed into a suitable enhancer tank or reactor where the feedstock is to be enhanced, the products from same (i.e., solid soil amendments 326 and liquid soil amendments 330), may be sold as nutrient rich amendments for soil. The organic soil enhancers 280 of the present invention efficiently and synergistically in some instances converts the received feedstock into nutrient rich soil-amendments. By way of example, for every ton of organic waste processed (i.e. food waste 18 and/or agricultural waste 19 via anaerobic digestion 46) there is produced about 440 pounds of solid digesate 58 and about 145 gallons of liquid digestate 54, which can be used as organic soil amendments/enhancers 280.

Similarly, after adding an appropriate, particular, desired amount (one ton) (a stoichiometric amount in certain instances) of one or more of seaweed 310, algae 314 and animal waste 318, compost 290, peat 294, manure 298, and slurry 302, and mixtures thereof (feed stock enhancers) produces from about 300 lbs to about 600 lbs, more preferably from about 400 lbs to about 550 lbs, and most preferably from about 420 lbs to about 460 lbs (e.g. 440 lbs)) of nutrient rich soil amendments. Similarly, for each ton of liquid digestate 58 processed after adding the appropriate, particular and desired amount (a stoichiometric amount in certain instances) of feed-stock enhancer (e.g., one or more of seaweed 310, algae 314 and animal waste 318, compost 290, peat 294, manure 298, and slurry 302, and mixtures thereof) produces from about 100 gallons to about 400 gallons, more preferably from about 200 gallons to about 300 gallons, and most preferably from about 100 gallons to about 200 gallons (e.g. 145 gallons)) of a nutrient rich soil amendment. The particular enhancer(s) and amount(s) of same, depends on the desired, appropriate quality and amount of the final nutrient-rich soil/liquid amendment. Both the liquid and solid digestates (54 and 58 respectively) and the feedstock items for organic soil enhancers 280 (i.e. compost 290, peat 294, manure 298, slurry 302, seaweed 310, algae 314 and animal wastes 318) are usually given a mimimum residence time of 15 days. Residence time refers to the amount of time the organic material spends in either an oxygen-free environment, or a specialized tank designed to maximize the nutrient release resulting from the biochemical breakdown of the organic matter. The produced liquid soil amendments 330 and solid soil amendments 326 have improved physical properties (such as water retention, permeability, water infiltration, drainage, aeration and structure) over any feedstock (e.g. by way of example only, solid/liquid digestates, coal char) prior to adding the feed stock enhancers.

Referring now to FIGS. 1, 3 and 11, for describing an embodiment of the anaerobic digestion process 46, waste to be anaerobically digested is flowed into the anaerobic digester (tank) 46. For the embodiment of the inventions shown in FIGS. 1 and 3, food waste 18 from the municipal solid waste (MSW) 14 and liquid substrates are conveyed or transported by vehicle 20 to a bio-waste conditioner 24 where the food waste 18 (and liquid substrates if applicable) and agricultural waste 19 (if applicable) are conditioned before deposited into anaerobic digester tank 46 for anaerobic digestion, which is a naturally occurring bio-chemical process. As shown in FIG. 11, food waste 18 may be accompanied by agriculture waste 19, and yard waste 22 which may flow through conduit 200 and diverted by valve 194 to flow through conduit 23 (dashed lines in FIG. 1) to conduit 604 where it is combined with food waste 18, as shown in FIG. 1. Anaerobic digester tank 46 is an air-tight tank for conducting anaerobic digestion in the absence of oxygen. In tank 46 bacteria (microorganisms) breaks down the food waste 18 (and agricultural waste 19, liquid substrates, and yard waste 22) into any desired product, such as biogas (e.g. methane, carbon dioxide and traces of other ‘contaminant’ gases) 50, and residual digestates (e.g., liquid 54 and solid digestates 58). A separator 48 is provided for separating the residual digestates into liquid digestate 54 and solid digestate 58. Liquid digestate 54 is a sludge or slurry produced by methanogenesis. Solid digestate 58 is a cellulose-based digestate produced during acidogenesis and originates from (but is not limited to) food which is fibrous in nature, such as vegetables (e.g., lettuce, spinach and the like). In an embodiment of the invention, without any further processing, the liquid digestate 54 may be applied directly to crops as a low analysis fertilizer 33, and the solid digestate 58 may be used “as is” for animal bedding 51, or organic compost 41 for horticulture applications, all as best illustrated in FIG. 11. Biogas 50 may be used to produce electric energy 66 and thermal energy 70.

In a preferred embodiment of the invention, for each ton of waste processed by the anaerobic digester tank 46, the conversion of waste (e.g., agricultural waste 19, food waste 18 and yard waste 22) by anaerobic digester tank 46 produces from about 180 gallons of nutrient rich liquid digestate to about 210 gallons of nutrient rich liquid digestate, preferably from about 115 gallons to about 175 gallons, more preferably from about 130 gallons to about 160 gallons, most preferably from about 140 gallons to about 150 gallons (i.e. about 145 gallons). In another preferred embodiment of the invention, for each ton of waste, the conversion of waste by anaerobic digester tank 46 produces from about 330 pounds of nutrient rich solid fiber digestate to about 540 pounds of nutrient rich solid fiber, preferably from about 380 pounds to about 490 pounds, more preferably from about 420 pounds to about 460 pounds, most preferably from about 430 pounds to about 460 pounds (i.e. about 440 pounds). The anaerobic digester will operate under, but is not limited to, thermophillic conditions, where digestion optimally takes place between about 120 to about 135 degrees F., or at elevated temperatures up to about 158 degrees F., where thermophiles are the primary microorganisms present.

The biogas 50 produced by tank 46 may be used directly for any purposes, such as cooking fuel, or it maybe subsequently sent by conduit 52 to a combined heat and power (CHP) generating plant 62 for generating and producing electrical energy 66 and thermal energy 70. Generating plant 62 employs a power station (shown in FIG. 3) to simultaneously generate electrical power 66 and thermal energy 70 which is heat that may be used as desired.

In a preferred embodiment of the invention, for each ton of waste processed by anaerobic digester tank 46, the electric energy 66 produced ranges from about 220 kWh of electric energy to about 420 KWh of electric energy, preferably from about 280 kWh of electric energy to about 485 KWh of electric energy, more preferably from about 315 kWh of electric energy to about 445 KWh of electric energy, most preferably from about 315 kWh to about 345 kWh of energy (i.e. about 330 kWh). For each ton of waste processed by anaerobic digester tank 46, the produced thermal energy 70 ranges from about 220 kWh of thermal energy to about 420 KWh of thermal energy, preferably from about 280 kWh of thermal energy to about 485 KWh of thermal energy, more preferably from about 315 kWh of thermal energy to about 445 KWh of thermal energy, most preferably from about 315 kWh to about 345 kWh of thermal energy (i.e. about 330 kWh).

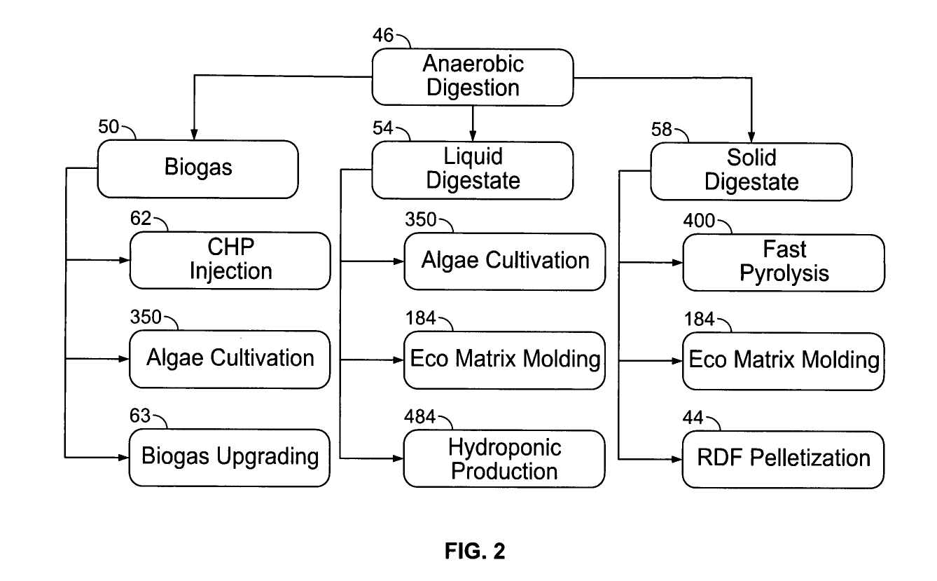

After the anaerobic digestion 46 produces biogas 50, the produced biogas 50 may be upgraded in the biogas upgrader 63 (apart from methane and carbon dioxide, biogas 50 can also contain water, hydrogen sulphide, nitrogen, oxygen, ammonia, siloxanes (silicon-oxygen bond compounds) and undesirable particulates). The concentrations of these impurities are dependent on the composition of the food waste 18 and the agricultural waste 19 and the anaerobic digestion process within tank 46 from which the biogas 50 was produced. Upgrading of biogas 50 may be conducted by any suitable means, such as by water washing, pressure swing absorption, selexol absorption, and amine gas treating. Biogas 50 may be used for CHP injection 62, algae cultivation 350 and biogas upgrading 63 (as best shown in FIG. 2).

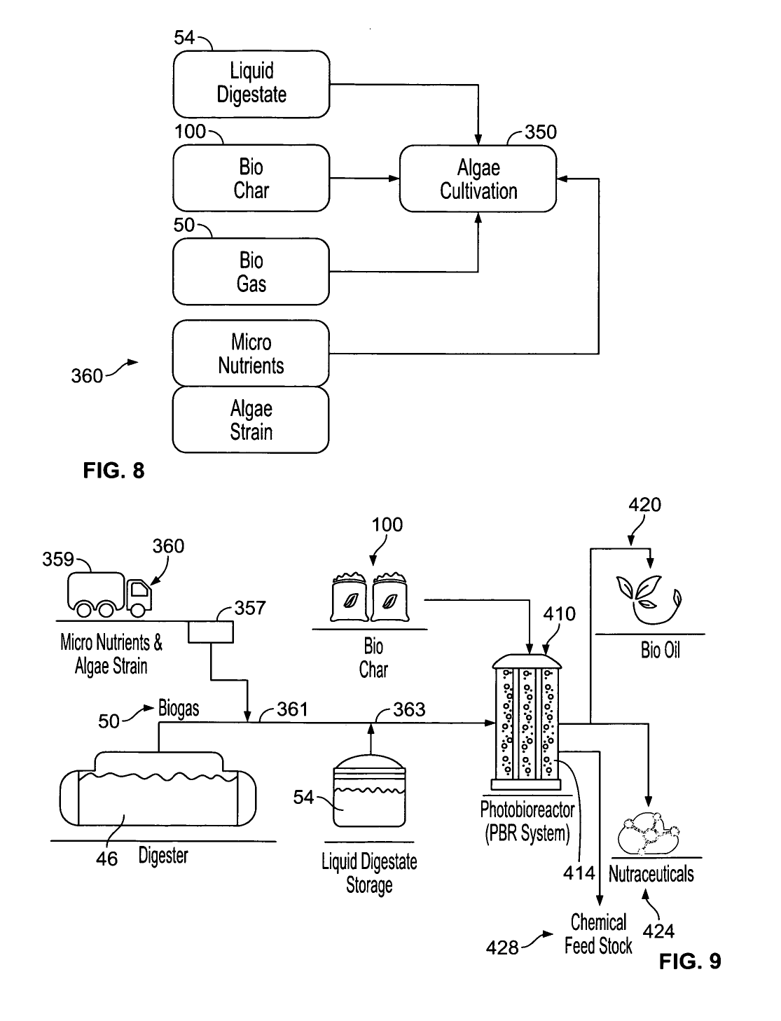

In another embodiment, as best shown in FIG. 1, a portion (or all) of the biogas 50 may be conveyed by conduit 56 to valve 74 where biogas 50 may be directed to flow through conduit 78 for algae cultivation 350. As best shown in FIG. 8, in addition to bio-gas 50, liquid digestate 54, and bio char 100, micro nutrients/algae strain 360 are employed. Liquid digestate 54 is provided to algae cultivation 350 by flowing through conduit 160, through valve 152, through conduit 164, through valves 190 and 86, through conduit 82, valve 74, and conduit 78. The bio char 100 is sent to algae cultivation 350 through a conduit 91, which is represented by dashed lines in FIG. 1.

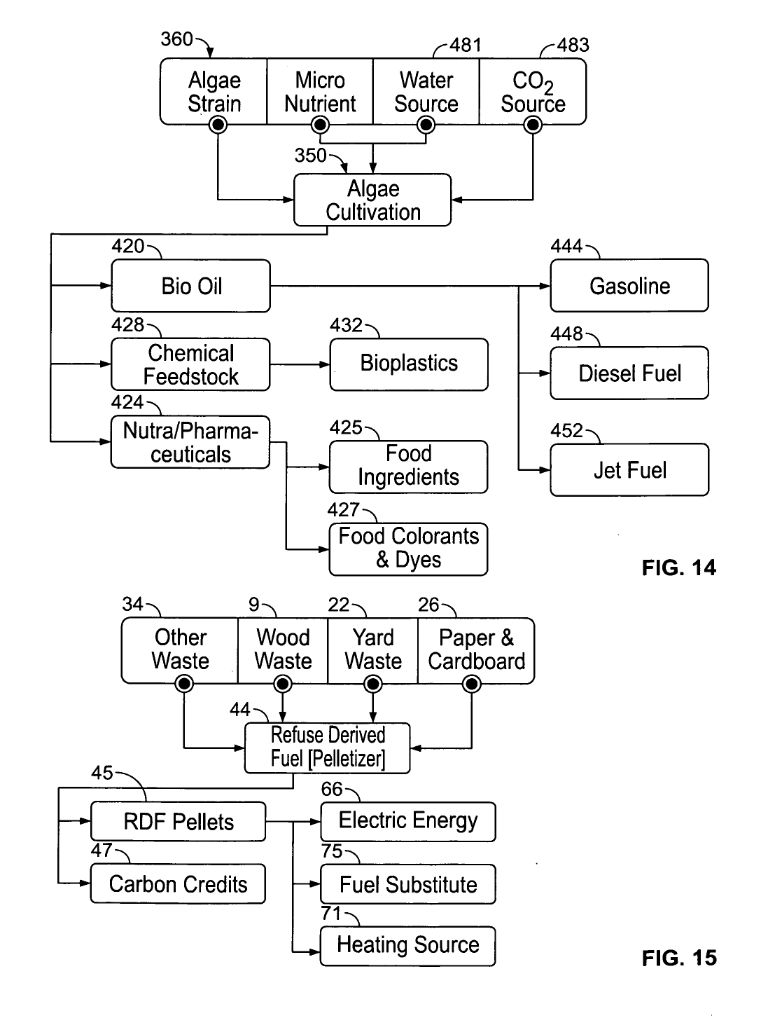

Referring now to FIG. 9 for a preferred embodiment of the invention, vehicle 359 delivers the micro nutrients/algae strain 360 to the algae-cultivation 350 location. Micro nutrients/algae strain 360 is stored in a tank 357. The growth of specialized algae strain requires the input of four main ingredients: the desired algae strain, micro nutrients (360 collectively), a water source 481, and a CO₂ source 483 (see FIG. 14). Commercial and industrial applications of the algae are numerous and include food ingredients such as omega-3 fatty acids or natural food colorants and dyes, fertilizer, bio-plastics additives, chemical feedstock, pharmaceuticals, and bio fuels. It is well known that micronutrients are nutrients required by humans and other organisms throughout life in small quantities to orchestrate a range of physiological functions.

Biogas 50 flowing from anaerobic-digestion tank 46 mixes with the micro nutrients/algae strain at juncture 361 from which the mixture flows toward juncture 363 where liquid digestate 54 is mixed with the mixture of biogas 50 and micro nutrients/algae strain 360. The mixture of the three components (i.e., micro-nutrients/algae strain, biogas 50 and liquid digestate 54) flow into a photo-bioreactor (PBR), generally illustrated as 410 in FIG. 9. As previously indicated, bio char 100 can also be used in the algae cultivation process 350, and is also introduced into the PBR 410, as best shown in FIG. 9. Ideal growing conditions cover three main criteria: (1) fluid velocity; (2) pH level; and (3) temperature. Optimal fluid velocity within the photobioreactor 410 is at a speed roughly equivalent, but not limited to about 1.60 feet per second—allowing for the necessary light and CO₂ absorption responsible for cultivation. With respect to pH, bubbling carbon dioxide rich gas through a photobioreactor (biogas 50) not only provides CO₂ to the algae, but also aids in deoxygenation of the suspension, provides mixing to increase the cycle frequency thereby limiting solar inhibition, and is even used to control pH. CO₂ may be consumed at a rate as high as about 26 g CO₂/m³-h. Optimal pH values for algae cultivation have also been reported to be between about 7.7 and about 8.5. Finally, temperature control plays an important role in growth propagation, and often heat exchangers using cold water have been shown to be effective for maintaining an optimal temperature range, which is between about 68 and about 86 degrees F.

The PBR 410 is a bioreactor that incorporates light source to provide photonic energy input into the reactor. Depending on facility location, PBR 410 can be designed to harness natural or artificial light. Three of the four feedstock sources for algae cultivation 350 are derived from the practice of embodiments of the invention during the anaerobic digestion phase 46: water, nutrients, and CO₂. Conventionally, algae cultivation systems connect or are coupled to industrial facilities in order to tap into outgoing CO₂ streams. In a preferred embodiment of the present invention, the biogas generated during anaerobic digestion phase 46 goes directly into the PBR 410 without having to tap into outgoing CO₂ streams. The liquid digestate 54 from the anaerobic digestion process 46 is nutrient rich, and can supplement a percentage of both water and additional nutritional needs of the system.

The configuration of PBR 410 lends itself to promoting biological growth efficiently through the control of key environmental parameters which include light, water flow, and nutrient injection. The tubes 414 of the PBR 410 are made of specialized materials designed to have both light and dark surface intervals, which enhances growth rate. Dedicated sensors (not shown) monitor aspects such as oxygen content, CO₂ release rate, fluid flow rate and harvesting rate. Once the algae is ready for harvesting, it is extracted via a filtering system (not shown) allowing only the desired sizes, while remaining algae passes back to the feeding assembly.

As shown in FIGS. 9 and 14, algae cultivation 350 via the PBR 410 produces bio oil 420, nutra/pharmaceuticals 424 (food containing health-giving additives and having medicinal benefit), and chemical feedstock 428 from which bio-plastics 432 (biodegradable plastic derived from biological substances) may be produced. The bio oil 420 may be upgraded by bio oil up-grader 440 (see FIG. 1) into gasoline 444, diesel fuel 448, and jet fuel 452. As best shown in FIGS. 1 and 14, nutralpharmaceuticals 424 may be converted in food ingredients 425 and food colorants/dyes 427. A branch represents the completion of a cell cycle and the algae cells dividing in two and the growth media doubling to maintain optimum cell density for photosynthesis.

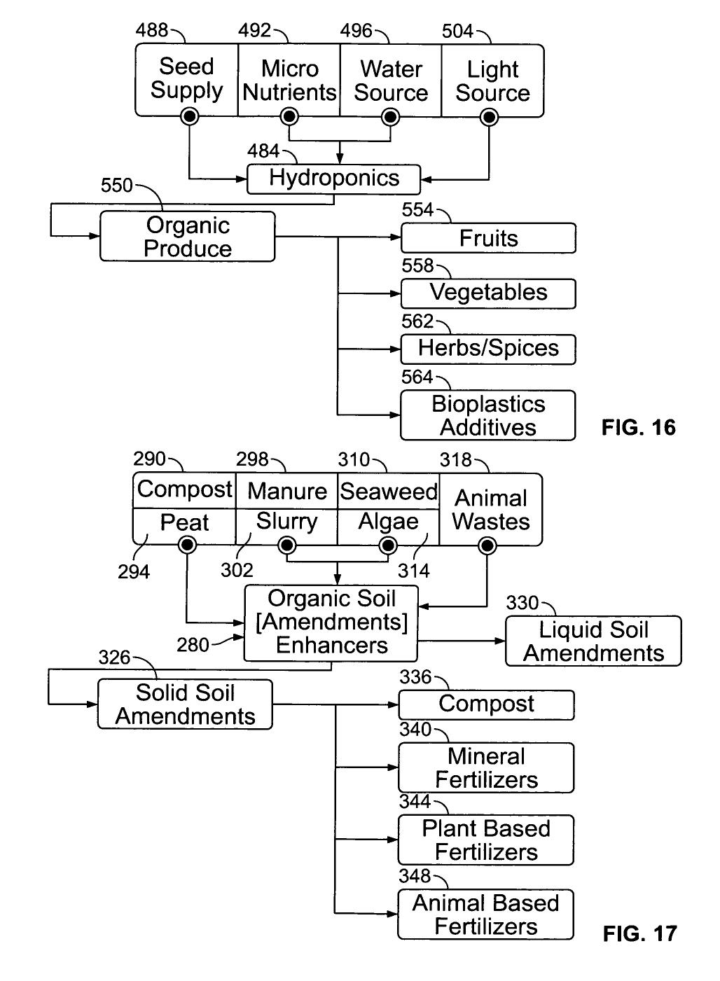

Referring now to FIGS. 1 and 16, there is seen the embodiment of the invention for cultivating organic produce in a liquid solution rather than in soil, i.e. hydroponics. The feedstock for the hydroponics system 484 comprises seed supply 488 from an outside vendor. The feedstock also includes micronutrients 492, water 496, and a light source 504 (see FIG. 16). Unlike traditional greenhouse or farm-based production, growing conditions are optimized in order to streamline energy efficiency and growth potential. Tying into existing feedstock availability from embodiments of the present invention, the nutrient rich liquid digestate 54 from the anaerobic-digestion tank 46 can be fed into the hydroponics system 484 for water (irrigation) 496 and as a source of micronutrients 492. The liquid digestate 54 produced by the anaerobic-digestion tank 46 is transmitted to the hydroponics system 484 through conduit 160, through valve 152, through valves 190 and 86, and conduit 542. Since the light source 504 provided to the plants is artificial, the electricity used to power the hydroponics 484 process is offset by the energy generated during the anaerobic digestion process 46 (electric energy 66). The hydroponics system 484 produces organic produce 550 which may comprise fruits 554, vegetables 558, herbs/spices 562 and bioplastics additives 564.

Lighting 504 is key in the growth of plants via hydroponics 484, and is provided artificially. Based on a typical Lumen Light chart, the optimal plant growth to light source relationship is within a distance of no more than two feet from the light source 504. Design of the hydroponic system 484 places plants within about 24 inches of the light source 504, thus achieving maximum lighting efficiency. Yet another attribute unique to the hydroponic system 484 is geotropism—the effect of gravity on plant growth hormones called auxins. By continually rotating plants horizontally top to bottom, these auxins are evenly distributed throughout the plant aiding in plant growth and strength.

The water source 496 lends itself to water conservation and plays a huge role in irrigation efficiency. In this hydroponic system 484 there is no runoff water, due to plant transpiration rates of about 440 to about 2,200 pounds of water for every pound of dry matter produced water use is decreased by about 95% per yield in comparison with traditional farming methods.

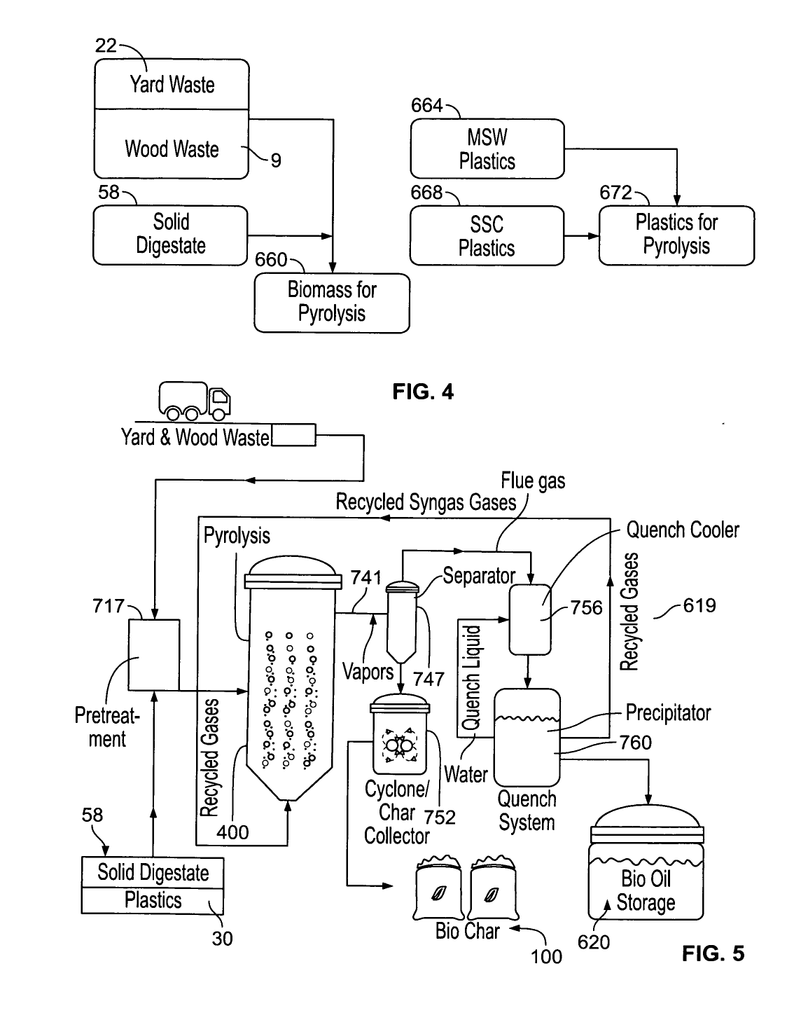

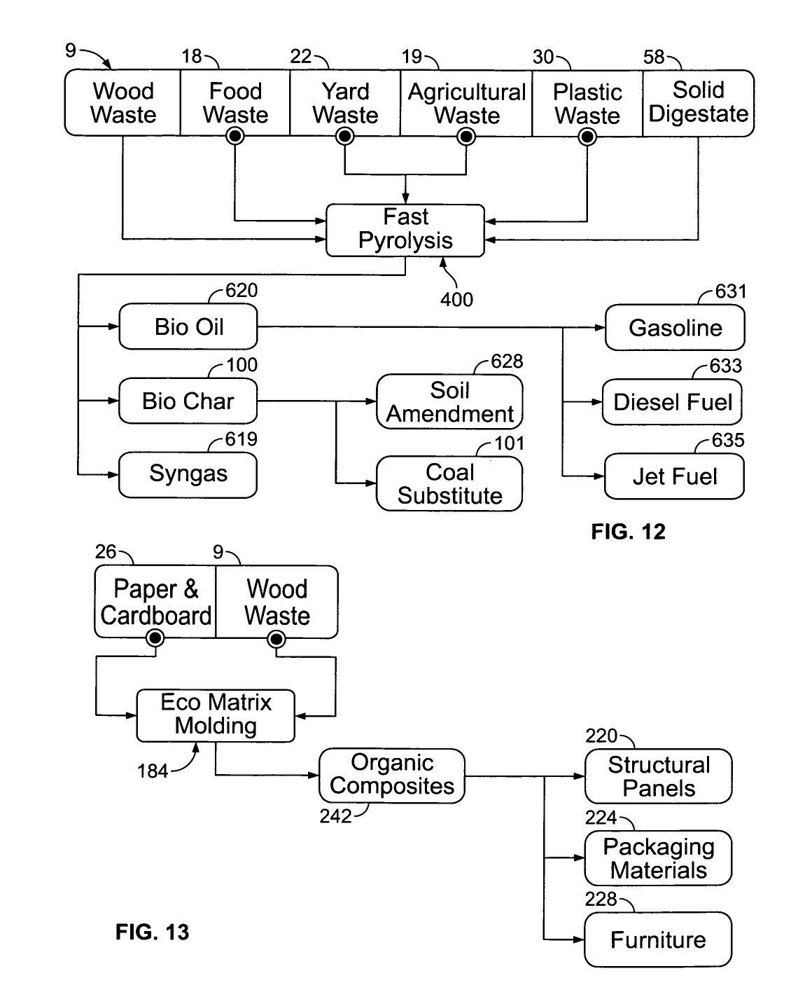

Referring now to FIGS. 1, 4, 5 and 12, there is illustrated an embodiment of the invention for conducting fast pyrolysis process in a fast pyrolysis plant, generally illustrated as 400. Pyrolysis produces biochar (charcoal) 100, bio oil 620, and synthetic gases 619 (as best shown in FIGS. 1, 5 and 12) from biomass by heating the biomass in a low/no oxygen environment. The absence of oxygen prevents combustion. Synthetic gas 619 may be used for process heating 696, as shown in FIG. 1. Bio char 100 may be used as a coal substitute 101, as well as organic soil amendment/enhancer 628 (referred in FIG. 1 as 280). As further shown in FIGS. 1 and 12, bio oil 620 may be upgraded by upgrader 627 into gasoline 631, diesel fuel 633, and jet fuel 635.

Varying from conventional operational schemes and as shown in FIG. 12, fast pyrolysis 400 for preferred embodiments of the present invention combine solid digestate 58, yard waste 22, wood waste 9, plastic waste 30 (SSC and MSW streams), food waste 18 and agricultural waste 19. The solid digestate 58 is one of the process derivatives resulting from the anaerobic digestion process 46. Solid digestate 58 flows through conduit 168, through valve 148 and then into fast pyrolysis 400. Yard waste 22 and wood waste 9 and plastic waste 30 feedstocks are each derived from a combination of source separated components (SSC) and municipal solid waste (MSW) streams. Yard waste 22 and wood waste 9 are procured from landscaping companies, agricultural partnerships and construction sites, while plastic waste 30 comes from a combination of pre-sorted recycling, industrial partnerships and traditional solid waste streams. Wood waste 9 and yard waste 22 of MSW 14 flow through conduit 200, through valves 194, 210, through conduit 200, through valve 190 and then into fast pyrolysis 400, as best shown in FIG. 1. As further best shown in FIG. 1 plastic waste 30 of MSW 14 flows through valve 210, through conduit 200, through valve 190 and subsequently into fast pyrolysis 400. Food waste 18 may be transferred to fast pyrolysis 400 by any suitable means.

In a preferred embodiment of the invention, for each ton of bio mass (e.g. food waste 18, agricultural waste 19, or any other biomass waste) processed by fast pyrolysis 400, the bio oil 620 produces ranges from about 10 gallons of bio oil 620 to about 90 gallons of bio oil, preferably from about 20 gallons to about 80 gallons, more preferably from about 30 gallons to about 70 gallons, most preferably from about 40 gallons to about 60 gallons (i.e. about 50 gallons). In another preferred embodiment of the invention, for each ton of bio mass processed by fast pyrolysis 400, the produced bio char 100 ranges from about 300 pounds of bio char 100 to about 500 pounds of bio char 100, preferably from about 330 pounds to about 470 pounds, more preferably from about 360 pounds to about 440 pounds, most preferably from about 390 pounds to about 410 pounds (i.e. about 400 pounds).

In a further preferred embodiment, for each ton of plastic waste 30 processed by fast pyrolysis 400, the produced bio char 100 ranges from about 300 pounds of bio char 100 to about 500 pounds of bio char, preferably from about 330 pounds to about 470 pounds, more preferably from about 360 pounds to about 440 pounds, most preferably from about 390 pounds to about 410 pounds (i.e. about 400 pounds). In yet another preferred embodiment of the invention, for each ton of plastic waste 30 processed by fast pyrolysis 400, the bio oil 620 produced ranges from about 120 gallons of bio oil 620 to about 150 gallons of bio oil 620, preferably from about 140 gallons to about 170 gallons, more preferably from about 150 gallons to about 180 gallons, most preferably from about 160 gallons to about 190 gallons (i.e. about 170 gallons).

As best shown in FIG. 5, secondary screening pretreatment apparatus 717 is applied for further purification, and the necessary mixtures of feedstock are created prior to injection into the fast pyrolysis plant 400. Upon completion of the preliminary sorting process, and pretreating process, all pyrolysis feedstock (e.g., biomass feedstock) sources are transported to the fast pyrolysis plant 400 where it/they are heated in a low/no oxygen environment. Exiting from the pyrolysis plant 400 are vapors 741 which enter into separator 747 where the vapors are separated into two streams, with one stream passing into a cyclone/char collector 752 to produce char 100, and with the other stream—flue gas—entering into a quench cooler 756 where flue gas is quenched and subsequently introduced into precipitator 760 which separates quench liquid (i.e., water) from synthetic gas 619, and bio oil 620. Quench liquid is recycled back to quench cooler 756. The synthetic gas 619 is recycled back to the pyrolysis plant 400 to provide a heating source (processing heating 696) to the pyrolysis plant 400. A closed loop recycled syngas system is provided and maintained. Employing, but not limited to, a fluidized bed system, this process is done under pressure and at operating temperatures above about 806 degrees F. For agricultural waste 19 (and yard waste 22 and wood waste 9), for example, typical temperatures range from about 840 to about 1,000 degrees F. Reactor pressure is kept at atmospheric pressure and no water is required in the process. After the feedstock has been heated, it is rapidly condensed within about 2 seconds. Furthermore, the maximum particle size is from about 1/16 inch to about ⅛ inch (in one dimension), and moisture content must be no more than about 10%.

Practice of preferred embodiments of the present invention strategically increases the lifecycle of each of the anaerobic digestion 46 process outputs (i.e. biogas 50, liquid digestate 54 and solid digestate 58); as opposed to not obtaining the outputs from anaerobic digestion 46, but from sources separate from anaerobic digestion 46. The lifecycles are increased by using them as feedstock sources for subsequent processes, all of which operate at a single facility (as best shown in FIG. 2). Biogas 50 produced by anaerobic digestion 46 follows three strategic pathways: (1) CHP injection 62; (2) biogas upgrading in biogas upgrader 63; and (3) algae cultivation 350. The CHP unit 62 produces electric and thermal energies 66 and 70, which are used to offset the facility's requirements (any excess is sold back to the grid). The secondary pathway places the biogas 50 through an upgrading process in biogas upgrader 63 which, separate from electric and thermal energies 66 and 70, can be used to produce fuels needed for transportation or movement of vehicles [e.g. cars] to transport or travel.

In algae cultivation 350, biogas 50 is pumped through conduit 56, through valve 74 and conduit 78 and serves as a growth catalyst for algae strains, which are capable of producing bio fuels (e.g. bio oil 420, chemical feedstock 428) and/or nutraceutical powders 424. In addition to use as a nutrient rich soil amendment, the liquid digestate 54 lifecycle is multiplied from a number greater than one (1) to about 5, preferable from about a number greater than one (1) to a number of about four (4), more preferable from a number greater than two (2) to a number of about four (4), e.g. from about 2.5 to about 3.5 (i.e. about 3) as it is re-purposed for applications such as in algae cultivation 350, hydroponics 484 and eco matrix molding 184.

For each ton of total waste (e.g., agricultural waste 19, food waste 18, yard waste 22, wood waste 9, etc.) processed by anaerobic digestion 46 for purposes of algae cultivation 350, hydroponics 484, fast pyrolysis 400, RDF 44, and eco matrix molding 184, produced liquid digestate ranges from about 180 gallons of liquid digestate to about 210 gallons of liquid digestate, preferably from about 115 gallons to about 175 gallons, more preferably from about 130 gallons to about 160 gallons, most preferably from about 140 gallons to about 150 gallons (i.e. about 145 gallons). For, large scale facilities, there will typically be a surplus required for downstream applications.

Algae cultivation 350, along with eco matrix molding 184, typically require an injection of water in which the algae strains can proliferate and the molds can be poured, respectively. Additionally, the nutrient rich liquid/water solution can be applied as a food source for the growth of hydroponic produce in hydroponic production 484. The use of water (more specifically liquid digestate 54) resulting from anaerobic digestion 46 can sustainably offset a percentage of downstream water needs. The amount of water resulting from anaerobic digestion 46 depends on the needs of downstream applications.

Similarly, the solid digestate 58 becomes a feedstock source for several downstream applications which include biomass pyrolysis 400, eco matrix molding 184, and RDF pelletization 44. Availability is abundant with the anaerobic digestion 46 process for preferred embodiments of the present invention. For each ton of waste (e.g., agricultural waste 19, food waste 18, yard waste 22, wood waste 9, etc.) processed by anaerobic digestion 46 for purposes of biomass pyrolysis 400, eco matrix molding 184, and RDF pelletization 44, the conversion of waste by anaerobic digester tank 46 produces from about 330 pounds of solid digestate to about 540 pounds of solid digestate, preferably from about 380 pounds to about 490 pounds, more preferably from about 420 pounds to about 460 pounds, most preferably from about 430 pounds to about 460 pounds (i.e. about 440 pounds). For, large scale facilities, there will typically be a surplus required for downstream applications.

For fast pyrolysis 400, the solid digestate 58 from the anaerobic digestion 46 is combined with yard waste 22, wood waste 9 and plastic waste 30 and fed into a fast-pyrolysis reactor 400 in order to increase the output of bio oil 620, bio char 100 and syngas 619. The solid digestate 58 from the anaerobic digestion 46 is a catalyst for fast pyrolysis 400. The eco matrix molding 184 offers feedstock flexibility. While core inputs for eco matrix molding 184 include paper and cardboard wastes 26 and wood waste 9 and yard waste 22, solid digestate 58 from the anaerobic digestion 46 can be mixed with the base components (i.e., paper and cardboard wastes 26, wood waste 9, and yard waste 22) in the generation of improved custom organic composites 242. Again, the solid digestate 58 from the anaerobic digestion 46 is an additive for eco matrix molding 184. Finally, any leftover solid digestate 58 is pelletized into RDF (refuse derived fuel) 44.

Reference throughout this specification to “one embodiment”, “an embodiment”, or “a specific embodiment” means that a particular feature, structure, or characteristic described in connection with the embodiment is included in at least one embodiment of the present invention and not necessarily in all its embodiments. Therefore, the respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment of the present invention may be combined in any suitable manner with one or more other embodiments. It is to be understood that other variations and modifications of the embodiments of the present invention described and illustrated herein are possible in light of the teachings herein and are to be considered as part of the spirit and scope of the present invention.

Additionally, any arrows in the drawings/figures should be considered only as exemplary, and not limiting, unless otherwise specifically noted. Furthermore, the term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

The foregoing description of illustrated embodiments of the present invention, including what is described in the Abstract, is not intended to be exhaustive or to limit the invention to the precise forms disclosed herein. While specific embodiments of, and examples for, the invention are described herein for illustrative purposes only, various equivalent modifications are possible within the spirit and scope of the present invention, as those skilled in the relevant art will recognize and appreciate. As indicated, these modifications may be made to the present invention in light of the foregoing description of the illustrated embodiments of the present invention and are to be included within the spirit and scope of the present invention.

Therefore, while the present invention has been described herein with reference to the particular embodiments thereof, a latitude of modification, various changes and substitutions are intended in the foregoing disclosures, and it will be appreciated that in some instances some features of the embodiments of the invention will be employed without the corresponding use of other features without departing from the scope and spirit of the invention as set forth. Therefore, many modifications may be made to adapt a particular situation or material to the essential scope and spirit of the present invention. It is intended that the invention not be limited to the particular terms used in following claims and/or to the particular embodiment disclosed as the best mode contemplated for carrying out this invention, but that the invention will include any and all embodiments and equivalents falling within the scope of the appended claims.

1-20. (canceled)

21. An apparatus to support and position the head or neck of a human being in the flexion position or the extension position,
comprising:
a platform to support said head or neck,
a support to support said platform,
a connection with a first part and a second part,
means to position said first part of said connection at or near one end of said platform,
means to position said second part of said connection at or near an end or side of said support,
means to mate said first part of said connection and said second part of said connection,
a mechanism to tilt said platform to position said head or neck in a flexion position or extension position.

22. An apparatus as defined in claim 1, wherein said platform is secured in said position.

23. An apparatus as defined in claim 1, wherein part or all of said platform is configured to mate with part or all of said head or neck.

24. An apparatus as defined in claim 1, wherein the result of the tilt of said platform is identified, and may be compared to a previous result.

25. An apparatus as defined in claim 1, further comprising: a pair of movable sidewalls.

26. An apparatus as defined in claim 25, wherein said pairs of movable sidewalls move inward toward the center of said platform.

27. An apparatus to position the neck of a human being in the flexion position or the extension position for taking medical pictures,
comprising:
a platform with ability to support said neck,
a support to support said platform,
an attachment that has at least a first part and a second part,
means to position said first part of said attachment at or near one end of said platform,
means to position said second part of said attachment at or near an end or side of said support,
means to mate said first part with said second part,
a mechanism to tilt said platform to position said neck in a flexion position or an extension position.

28. An apparatus as defined in claim 27, wherein said platform is secured in said position.

29. An apparatus as defined in claim 27, wherein part or all of said platform is configured to mate with at least part of the back of the head or neck of said human being.

30. An apparatus as defined in claim 27, wherein the result of the tilt of said platform is shown in terms related to degrees of angulation to a horizontal plane. and may be compared to a previous result.

31. An apparatus as defined in claim 27, further comprising: a pair of movable sidewalls.

32. An apparatus as defined in claim 31, wherein said pairs of movable sidewalls move inward toward the center of said platform.

33. An apparatus to support and position the head of a human being in the flexion position for taking medical pictures,
comprising:
a platform to support and position said head,
a support to support said platform,
an attachment method with a first part and a second part,
means to position said first part at or near one end of said platform,
means to position said second part at or near an end or side of said support,
means to mate said first part with said second part to attach said platform and said support,
a connection to locate said support at or near one end of a medical examining table,
a mechanism to tilt said platform to position said head in a flexion position.

34. An apparatus as defined in claim 33, wherein said platform is secured in said position.

35. An apparatus as defined in claim 33, wherein part or all of said platform is configured to mate with at least part or all of the back of said head.

36. An apparatus as defined in claim 33, further comprising: a pair of movable sidewalls.

37. An apparatus as defined in claim 36, wherein said pairs of movable sidewalls move inward toward the center of said platform.
